(19) United States
(12) Patent Application Publication
(10) Pub. No.: US 2018/0296720 A1
(43) Pub. Date: Oct. 18, 2018

(54) FUNCTIONAL PATCH BENEFICIAL TO HUMAN BODY FOR PAD

(71) Applicant: DONG HAE DIAPER CO., LTD., Gangwon-do (KR)

(72) Inventor: Jun Hwan IM, Seoul (KR)

(21) Appl. No.: 15/758,817

(22) PCT Filed: Aug. 16, 2016
(86) PCT No.: PCT/KR2016/008965

§ 371 Date: Mar. 9, 2018

(30) Foreign Application Priority Data
Sep. 17, 2015 (KR) 10-2015-0131549

(51) Int. Cl. A61L 15/18; C04B 35/26; C04B 35/626; C04B 35/64; C04B 35/622; B41M 1/26

(52) U.S. Cl. CPC A61L 15/18; C04B 2235/6567; C04B 35/62695; C04B 35/64; C04B 35/6262; C04B 35/6263; C04B 35/62222; B41M 1/26; A61L 2420/04; C04B 2235/3274; C04B 2235/349; C04B 2235/3418; C04B 2235/3481; C04B 2235/3472; C04B 2235/442; C04B 2235/3232; C04B 2235/3217; C04B 2235/3244; C04B 2235/6026; C04B 35/2616

(57) ABSTRACT

A functional patch for a pad is beneficial to a human body and solves the problems entailed in various sanitary pads having a multi-layer configuration such as a sanitary napkin, a diaper for an infant or an adult, a sanitary sheet, a mask, etc. The functional patch is inserted into and attached to the pad formed in a multi-layer. Thus, functionality of a sanitary pad may be improved.

### TECHNICAL FIELD

The present invention relates to a functional patch for a pad beneficial to a human body which is made of a mixture of ferrite sintered body and ceramic sintered body, and more particularly, to a functional patch for a pad, which is beneficial to a human body and is formed by mixing a raw material for ferrite sintered body and a raw material for ceramic sintered body in a desired mixing ratio, and then, printing a functional composition obtained from the above mixture on an upper surface of a fabric such as a non-woven fabric to form a patch, so as to absorb or neutralize electromagnetic waves and water vein waves and to achieve effects of natural terrestrial magnetism generation, effects of anion and far-infrared radiation, and anti-microbial and deodorization effects when the prepared functional patch is inserted and attached inside a sanitary pad such as a sanitary napkin, a diaper for an infant or an adult, a sanitary sheet, a mask, etc., thereby improving health conditions of a person wearing the sanitary pad.

### BACKGROUND ART

A sanitary pad to which the present invention is applied may include, for example, a sanitary napkin, a diaper for an infant or an adult, a sanitary sheet for a patient kept in bed for a long time, and a mask made of multiple layers. Common features of the above-listed sanitary pads are that they include fabrics formed in a multi-layer, and are put on a body and used for long hours. Therefore, as suggested in the title of invention, the sanitary pad should satisfy conditions beneficial to health of the human body.

The sanitary napkin described above is a daily necessity for adult women in order to absorb secretion such as menstrual blood and leucorrhea. Considering that the sanitary napkin is an item closely used by women as a daily necessity, a problem of this item is that an unpleasant feeling is often caused by a bad smell of secretion reeking during absorption thereof. Further, the sanitary napkin is continuously maintained in a wet state due to mucous secretion to induce conditions susceptible to growth of bacteria and molds, thus causing itching. Further, the skin surrounding the private parts (such as genitals or vaginal part) closely contacting the sanitary napkin becomes sore or festering.

Similarly, the diaper for an infant or an adult has also a problem that the private parts are maintained in a wet state due to secretion or excrement and, if a user wears the diaper for a long time, often encounters foul smell and skin troubles such as itching. Further, in a case of the sanitary sheet for a patient kept in bed for a long time, a part contacting the skin of the patient becomes wet to smell bad. Furthermore, in a case of the mask for a mouth, saliva and breath discharged from the mouth are directly adsorbed to the mask while wearing the mask over the mouth, and therefore, problems such as growth of noxious bacteria as well as bad smell are encountered when wearing the mask for a long time.

### DISCLOSURE

### Technical Problem

Accordingly, the present invention has been made to solve the problems entailed in various sanitary pads having a multi-layer configuration such as a sanitary napkin, a diaper for an infant or an adult, a sanitary sheet, a mask, etc., and it is an object of the present invention to provide a sanitary patch for a pad, which is beneficial to a human body, thereby improving functionality of a sanitary pad when this functional patch beneficial to a human body is inserted into a pad formed in a multi-layer and attached thereto.

The functional patch for a pad according to the present invention, which is beneficial to the human body, may be formed by: mixing a ferrite sintered body, which absorbs and neutralizes electromagnetic waves and water vein waves, and achieves effects of terrestrial magnetism generation, and a ceramic sintered body which achieves effects of anion and far-infrared radiation and anti-microbial and deodorization effects; adding a natural mordant to the mixture; and then, printing the obtained mixture onto a fabric such as a non-woven fabric in a constant pattern, thus to form, a patch type product.

### Technical Solution

According to one aspect of the present invention, there is provided a functional patch for a pad, which is beneficial to a human body and is formed through a series of processes including: a ferrite mixing process which comprises mixing 45-55% by weight (wt. %) of ferric oxide, 12-16 wt. % of nickel oxide, 14-18 wt. % of zinc oxide, 4-6 wt. % of copper oxide, 4-8 wt. % of lithium, 4-8 wt. % of barium and 2-4 wt. % of magnesium to prepare a mixed ferrite raw material (S110); a suspending process of a ferrite raw material, which comprises introducing 130-170 wt. % of water, 2-4 wt. %, of a dispersant and 2-3 wt. % of a binder relative to a weight of the mixed raw material, respectively, into the mixed raw material to prepare a mixture in a suspension state (S120); a granulation process of a ferrite raw material, which comprises dry-spraying the mixture in a suspension state to form a granular shape (S130); a heat treatment and sintering process of heating the granular ferrite raw material at a temperature of 1,000-1,400° C. for 3-8 hours to calcine a ferrite sintered body (S140); a powdering process of the heat-treated ferrite sintered body to pulverize the same into powders (S150); a liquefaction process of the ferrite sintered body, which comprises mixing 40-60 wt. % of the powdered ferrite sintered body with 40-60 wt. % of water, adding 2-6 wt. % of a natural mordant to a weight of the mixture of ferrite powders and water, and ageing the same for 12-24 hours (S160); and a printing process of printing the liquefied ferrite sintered body onto a fabric in predetermined design and pattern (S170).

### Advantageous Effects

When the functional patch for a pad according to the present invention, which is beneficial to the human body, is inserted into a sanitary pad such as a sanitary napkin, a diaper for an infant or an adult, a sanitary sheet and a mask and attached thereto, it is possible to attain advantages of overcoming problems such as itching, bad smell, bacterial growth, skin troubles, etc. that possibly occur while wearing the sanitary pad. In particular, for a sanitary napkin, it is possible to alleviate menstrual pain and promote blood circulation, thus to remarkably reduce an intrauterine disease, in addition to removal of bad smell, thereby achieving effects of improving a healthy life.

Meanwhile, the functional patch of the present invention may protect modern people exposed defenselessly to various harmful electromagnetic waves from the water vein waves as well as the electromagnetic waves. Further, the functional patch of the present invention may substantially enjoy effects of terrestrial magnetism generation, effects of anion and far-infrared radiation, and anti-microbial and deodorization effects closely in our daily life, thereby enabling realization of a healthy life.

### BRIEF DESCRIPTION OF DRAWINGS

FIG. 1 is a schematic view illustrating a functional patch of the present invention and various sanitary pads.

FIG. 2 is a view showing a use state of the functional patch according to the present invention, which is applied to a sanitary napkin.

FIG. 3 is a schematic view illustrating functions of the sanitary napkin to which the functional patch according to the present invention is attached.

FIG. 4 is a flow chart illustrating manufacturing processes of a functional patch for a sanitary pad, which is made of a ferrite sintered body according to the present, invention.

FIG. 5 is a flow chart illustrating manufacturing processes of a functional patch for a sanitary pad, which is made of a ceramic sintered body according to the present invention.

FIG. 6 is a flow chart illustrating manufacturing processes of a functional patch for a sanitary pad, which is made of a mixture of ferrite sintered body and ceramic sintered body.

### BEST MODE

Hereinafter, technical configurations and effects of preferred embodiments of the present invention to accomplish the purposes of the present invention will be described with reference to the accompanying drawings. However, if physicochemical techniques known in the art in relation to the present invention and/or conventionally known art in regard to printing and coating processes are nothing more than a supporting detail which does not match with the concept of the present invention, detailed description thereof would be omitted.

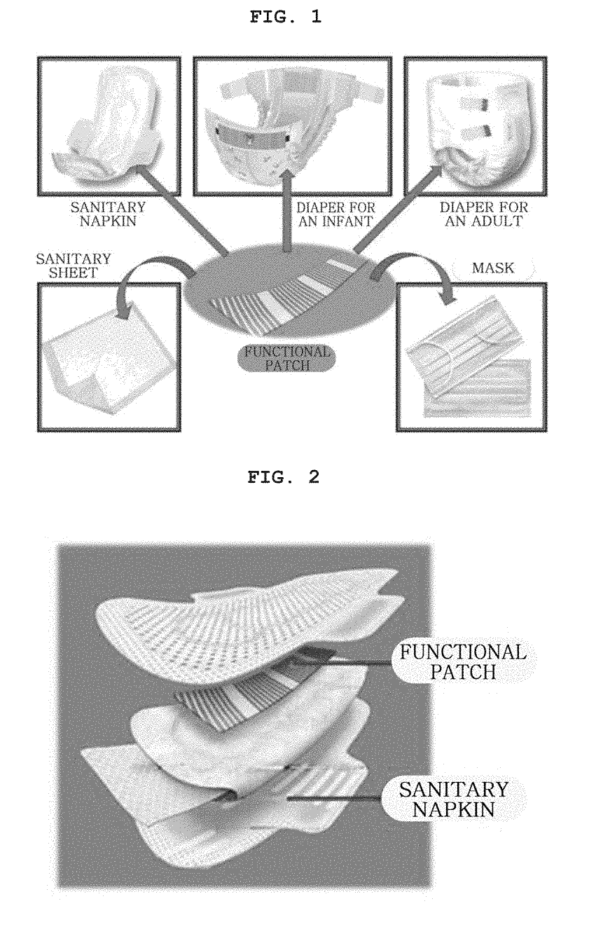

FIG. 1 is a schematic view illustrating a functional paten of the present invention and various sanitary pads. As illustrated in FIG. 1, common features of a sanitary napkin, a diaper for an infant or an adult, a sanitary sheet for a patient and a mask are that they include fabrics formed in a multi-layer and are used as a sanitary pad closely put on the human body.

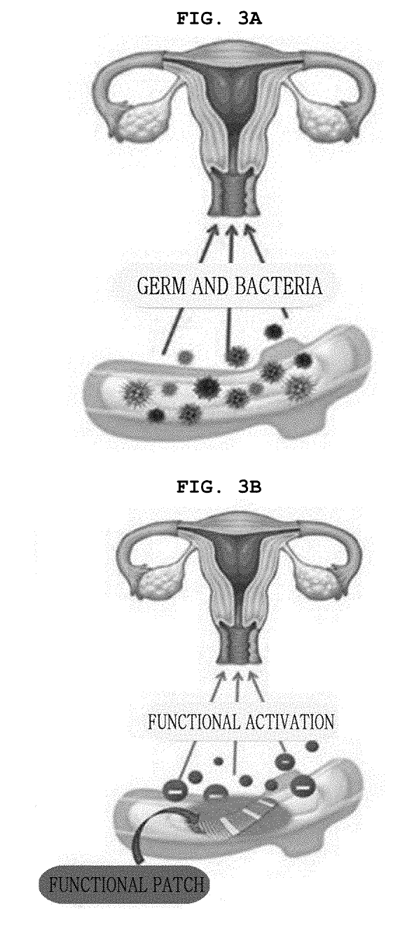

Considering that conventional sanitary napkins are used as a pad to absorb secretion such as menstrual blood and leucorrhea, unpleasant feeling may be increased due to bad smell reeking from, the secretion during absorption thereof. Further, a wet state is continued due to mucous secretion, thereby causing a condition easy for forming bacteria and molds, as shown in FIG. 3A. As a result, itching, etc. occurs and the skin surrounding the private parts closely contacting the sanitary napkin often becomes sore or festering. Moreover, there is a problem of causing intrauterine diseases.

Meanwhile, in a case of a diaper for an infant or an adult, a wet state is caused due to secretion or excrement from the private parts. Therefore, wearing the diaper for a long time encounters problems such as bad smell, itching, skin troubles, etc. Further, in a case of a sanitary sheet for a patient, a wet condition occurs at a part contacting the skin to generate bad smell. Furthermore, in a case of a mask for wearing over a mouth, saliva or breath discharged from the mouth is directly adsorbed to the mask over the mouth during wearing, thus causing problems such as growth of harmful bacteria and bad smell.

Accordingly, as shown in a center of FIG. 1, when inserting the functional patch beneficial to the human body according to the present invention into a variety of sanitary pads and attaching thereto, the above-described problems may be overcome.

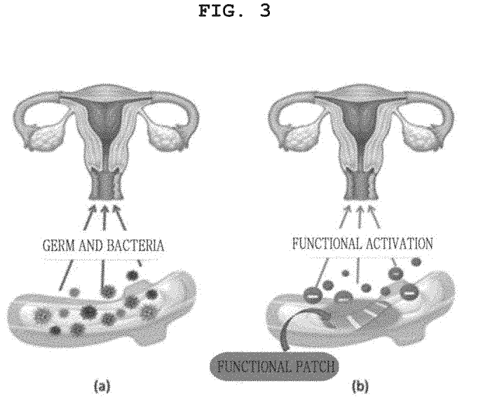

For example, FIG. 2 is a view showing a use state of the functional patch according to the present invention, which is applied to a sanitary napkin, and FIG. 3 is a schematic view illustrating functions of the sanitary napkin to which the functional patch according to the present invention is attached. As shown in FIG. 2 or FIG. 3B, when inserting the functional patch into the sanitary napkin formed in a multi-layer, it is possible to protect modern people who are exposed defenselessly to various electromagnetic waves, from water vein waves as well as the electromagnetic waves. Further, effects of terrestrial magnetism generation, effects of anion and far-infrared radiation, and anti-microbial and deodorization effects are attained from the base inside the sanitary napkin. As a result, itching and festering at the surrounding skin of the private parts while wearing the sanitary napkin may be resolved, bacteria growth may be prevented, menstrual pain may be relieved, and blood circulation may be promoted, thus to remarkably reduce intrauterine diseases, thereby improving a healthy life.

In addition, the functional patch of the present invention has a function of absorbing and neutralizing electromagnetic waves. Further, due to rapid development in wired and wireless communications technique and explosive popularization of personal mobile communications, modern people are currently exposed defenselessly to electromagnetic waves even though not carrying a portable terminal.

Accordingly, in recent, years, people suffering from a disease such as video display terminal (VDT) syndrome have been increased. The above-described VDT syndrome refers to a disease of modern times, which, occurs due to a thermal action caused by a microwave used in a portable communication terminal, a microwave oven, etc. or due to some symptoms such as headache, visual disturbance, etc. caused by electromagnetic waves generated from a computer, a monitor, and the like.

As described above, in order to improve a healthy life of modern people exposed defenselessly to the electromagnetic waves harmful to the human body, the functional patch according to the present invention has been developed to impart effects of the functional patch close to the human body by means of a sanitary pad.

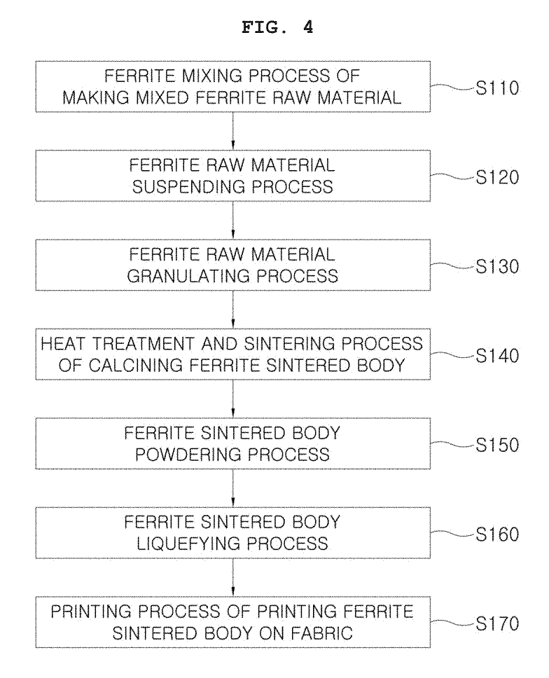

FIG. 4 is a flow chart illustrating manufacturing processes of a functional patch for a sanitary pad, which is made of a ferrite sintered body according to the present invention. A first step of the present invention includes measuring raw materials contained in the ferrite sintered body and mixing the same in a predetermined ratio by weight.

That is, the raw materials for forming the ferrite sintered body of the present invention may be mixed in a mixing ratio by weight, in particular, 45-55% by weight (wt. %) of ferric oxide, 12-16 wt. % of nickel oxide, 14-18 wt. % of zinc oxide, 4-6 wt. % of copper oxide, 4-8 wt. % of lithium, 4-8 wt. % of barium and 2-4 wt. % of magnesium, are mixed together (S110, a ferrite mixing process to prepare a mixed ferrite raw material).

A second step in the manufacturing of a functional patch using the ferrite sintered body includes introducing 130-170 wt. % of water, 2-4 wt. % of a dispersant and 2-3 wt. % of a binder relative to a weight of the mixed raw material, respectively, which was prepared in the first step, into the above mixed raw material, and then, preparing a viscous mixture in a suspension state by a stirring device such as a ball mill (S120, a suspending process of a ferrite raw material to prepare a mixture in a suspension state),

The dispersant used herein may be added to uniformly disperse granules of the mixed raw material in water, thereby forming a high quality suspension, and may include, for example, ethoxylated alkanolamides.

Further, the binder may increase a binding force between raw material granules in a granulation process of a ferrite composition which is subsequently conducted, so as to easily fulfill granulation of ferrite. For example, a water-soluble solution of polyvinyl alcohol may be used as the binder.

A third step in the manufacturing of a functional patch using a ferrite sintered body includes dry-spraying the mixture in a suspension state to form a granular shape (S130, a granulation process of a ferrite raw material). The suspension prepared in the second step is carried in a constant container and injected through a nozzle. In this case, the suspension of ferrite raw material is agglomerated by a binder thus being granulated when the suspension is injected into a housing in a not air blow condition or in an atmosphere at a higher pressure.

Meanwhile, a fourth step in the manufacturing of a functional patch using a ferrite sintered body includes heating the granular ferrite raw material by a method such as furnace heating at a temperature of 1,000-1,400° C. for 3-8 hours to calcine a ferrite sintered body (S140, a heat treatment and sintering process of calcining a ferrite sintered body). Through the heat treatment in the fourth step, impurities such as a dispersant or binder remaining in the ferrite raw material may be removed.

A fifth step in the manufacturing of a functional patch using a ferrite sintered body includes pulverizing the heat-treated ferrite sintered body, which was obtained from the fourth step, into powders by a conventional grinding device (S150, a powdering process of pulverizing the ferrite sintered body into powders)

A sixth step in the manufacturing of a functional patch using a ferrite sintered body includes mixing 40-60 wt. % of the powdered ferrite sintered body and 40-60 wt. % of water, adding 2-6 wt. % of a natural mordant to a weight of the mixture of the ferrite powders and water, and ageing the same for 12-24 hours (S160, a liquefaction process of the ferrite sintered body).

That is, in order to manufacture a functional patch printed onto a fabric such as a non-woven fabric, a liquefaction process of mixing a natural mordant with the ferrite sintered body powdered in the previous process is conducted, wherein the mordant is added so as to clearly print design and pattern in a concentrated manner without being blur. According to the present invention, the natural mordant used herein may include apple juice, schisandra juice, tree barks, vegetable tannin, etc.

A seventh step in the manufacturing of a functional patch using a ferrite sintered body includes printing the liquefied ferrite sintered body, which was obtained from the sixth step, onto a fabric in predetermined design and pattern, thereby forming the functional patch (S170, a printing process of ferrite sintered body)

Considering that a specific printing technique of printing a functional mixture as described in the present invention onto a fabric such as a non-woven fabric is already well known in the art, and that a description of the above known technique is nothing more than a supporting detail which does not match with the concept of the present invention, the specific printing process will not be described in detail.

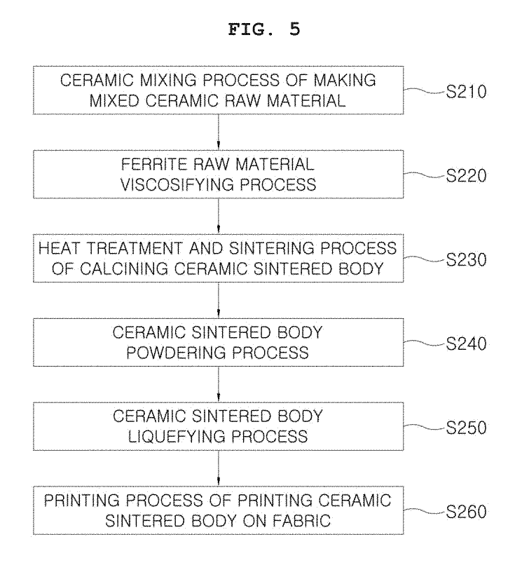

FIG. 5 is a flow chart illustrating manufacturing processes of a functional patch for a sanitary pad, which is made of a ceramic sintered body according to the present invention. The manufacturing processes of a functional patch using a ceramic composition to achieve anion, and/or far-infrared radiation effects beneficial to the human body, as well as anti-microbial and deodorization effects will be described in detail with reference to FIG. 5.

As illustrated in FIG. 5, a first step in the manufacturing of a functional patch using a ceramic sintered body includes measuring raw materials contained in the ceramic sintered body and mixing the raw materials in a predetermined ratio by weight.

That is, the raw materials for forming the ceramic sintered body of the present invention may be mixed in a mixing ratio by weight, in particular, 20-30 wt. % of elvan, 20-30 wt. % of tourmaline, 10-18 wt. % of feldstar, 10-18 wt. % of kaolin, 6-10 wt. % of rock material, 6-10 wt. % of white clay and 4-8 wt. % of sericite are mixed together (S210, a ceramic mixing process to prepare a mixed ceramic raw material).

The elvan in the mixed raw material of ceramic sintered body emits far-infrared rays beneficial to the human body, has deodorization and anti-microbial effects through an adsorption action, and activates physiological function due to ion radiation, thereby being effective for protection and treatment of diseases. Further, tourmaline increases activation of cells through action of anions, promotes smooth blood circulation, while the white clay emits far-infrared rays by itself to activate physiological function of cells, thereby detoxifying harmful materials contained in the cells of the human body.

Meanwhile, a second step in the manufacturing of a functional patch using a ceramic sintered body includes introducing 100-140 wt. % of water to a weight of the mixed raw material, introducing 5-7 wt. % of limestone, 4-6 wt. % of titanium oxide, 6-8 wt. % of aluminum oxide and 1-3 wt. % of zirconium to a weight of the mixed raw material, and then, heating the mixture at a temperature of 200-300° C. for 3-8 hours to prepare a viscous mixture in a slurry state (S220, a viscosifying process of the ceramic raw material to prepare a mixture in a slurry state).

Further, a third step in the manufacturing of a functional patch using a ceramic sintered body includes heating the slurry type mixture by means of furnace heating

at a temperature of 1,000-1,400° C. for 3-8 hours to calcine a ceramic sintered body (S230, a heat treatment and sintering process of calcining the ceramic sintered body). Through the heat treatment and sintering process in the third step, impurities remaining in the ceramic raw material may be removed.

A fourth step in the manufacturing of a functional patch using a ceramic sintered body includes pulverizing the heat-treated ceramic sintered body, which was obtained from the third step, into powders by a conventional grinding device (S240, a powdering process of the ceramic sintered body to pulverize the ceramic sintered body into powders).

Meanwhile, a fifth step in the manufacturing of a functional patch using a ceramic sintered body includes mixing 40-60 wt. % of the powdered ceramic sintered body, which was obtained from the fourth step, with 40-60 wt. % of water, adding 2-6 wt. % of a natural mordant to a weight of the mixture of the ceramic powders and water, and ageing the same for 12-24 hours (S250, a liquefaction process of the ceramic sintered body).

That is, in order to manufacture the functional patch printed onto a fabric such as a non-woven fabric, a liquefaction process of mixing a natural mordant with the ceramic sintered body powdered in the just previous process is conducted, wherein the mordant is added so as to clearly print design and pattern in a concentrated manner without being blur. According to the present invention, the natural mordant used herein may include apple juice, schisandra juice, tree barks, vegetable tannin, etc.

A sixth step in the manufacturing of a functional patch using a ceramic sintered body includes printing the ceramic sintered body, which was liquefied in the fifth step, onto a fabric in predetermined design and pattern to form a patch (S260, a printing process of the ceramic sintered body).

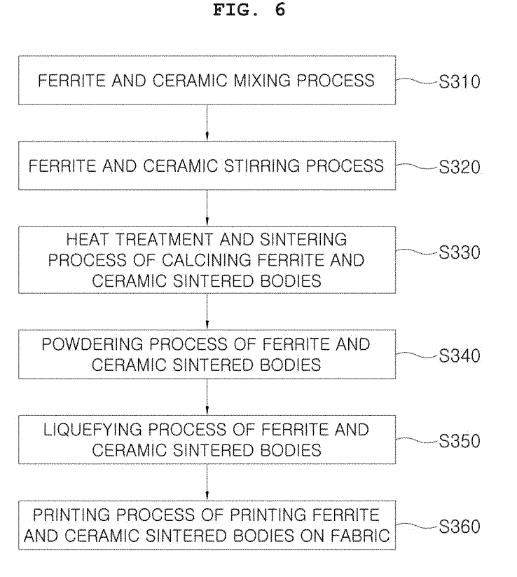

FIG. 6 is a flow chart illustrating manufacturing processes of a functional patch for a sanitary pad, which is made of a mixture of ferrite sintered body and ceramic sintered body (‘ferrite and ceramic sintered bodies’). With reference to FIG. 6, the following description will be given to explain the manufacturing processes of a functional patch for a pad using ferrite and ceramic mixed compositions, including: mixing a ferrite sintered body which absorbs and neutralizes electromagnetic waves and water vein waves harmful to the human body, and achieves effects of terrestrial magnetism generation, and a ceramic sintered body which achieves effects of anion and far-infrared radiation and anti-microbial and deodorization effects; adding a natural mordant to the mixture; and printing the mixture onto a fabric such as a non-woven fabric in predetermined design and pattern to manufacture a patch, thereby helping modern people to enjoy a healthy life.

As illustrated in FIG. 6, a first step in the manufacturing of a functional patch using ferrite and ceramic sintered bodies includes mixing 40-60 wt. % of the powdered ferrite composition and 40-60 wt. % of the ceramic powders (S310, a mixing process of ferrite and ceramic).

A second step in the manufacturing of a functional patch using ferrite and ceramic sintered bodies includes introducing 35-55 wt. % of water to a weight of the ferrite and ceramic mixture and forming a mixture in a paste state by means of a stirring device such as a ball mill (S320, a stirring process of ferrite and ceramic).

A third step in the manufacturing of a functional patch using ferrite and ceramic sintered bodies includes heating the stirred product of ferrite and ceramic in a paste state by means of furnace heating at a temperature of 1,000-1,500° C. for 10-14 hours to calcine the ferrite and ceramic sintered bodies (S330, a heat treatment and sintering process of calcining ferrite and ceramic sintered bodies), Through the heat treatment and sintering process in the third step, impurities remaining in the ferrite and ceramic raw materials may be removed.

A fourth step in the manufacturing of a functional patch using ferrite and ceramic sintered bodies includes pulverizing the heat-treated ferrite and ceramic sintered bodies into powders by a conventional grinding device (S340, a powdering process of ferrite and ceramic sintered bodies).

Further, a fifth step in the manufacturing of a functional patch using ferrite and ceramic sintered bodies includes mixing 40-60 wt. % of the ferrite and ceramic sintered bodies which were powdered in the fourth step, with 40-60 wt. % of water, adding 2-6 wt. % of a natural mordant to a weight of a mixture of the ferrite and ceramic powders and water, and then, ageing the same for 12-24 hours (S350, a liquefaction process of the ferrite and ceramic sintered bodies).

That is, in order to manufacture the functional patch printed onto a fabric such as a non-woven fabric, a liquefaction process of mixing a natural mordant with the ferrite and ceramic sintered bodies powdered in the just previous process is conducted, wherein the mordant is added so as to clearly print design and pattern in a concentrated manner without being blur. According to the present invention, the natural mordant used herein may include apple juice, schisandra juice, tree barks, vegetable tannin, etc.

A sixth step in the manufacturing of a functional patch using ferrite and ceramic sintered bodies includes printing the ferrite and ceramic sintered bodies, which were liquefied in the fifth step, onto a fabric in predetermined design and pattern to form a patch (S360, a printing process of ferrite and ceramic sintered bodies)

As a result of passing through the first step to the sixth step in the manufacturing of a functional patch using ferrite and ceramic sintered bodies, as described above, a functional patch for a pad, which is beneficial to the human body and is formed using a mixture of ferrite and ceramic sintered bodies, may be manufactured, thereby absorbing and neutralizing electromagnetic waves and water vein waves, and achieving effects of natural terrestrial magnetism generation, effects of anion and far-infrared radiation, and anti-microbial and deodorization effects.

1. A functional patch for a pad, which is beneficial to a human body and is formed through a series of processes comprising:
a ferrite mixing process which comprises mixing 45-55% by weight (wt. %) of ferric oxide, 12-16 wt. %) of nickel oxide, 14-18 wt. % of zinc oxide, 4-6 wt. % of copper oxide, 4-8 wt. % of lithium, 4-8 wt. % of barium and 2-4 wt. % of magnesium to prepare a mixed ferrite raw material;
a suspending process of a ferrite raw material, which comprises introducing 130-170 wt. % of water, 2-4 wt. %, of a dispersant and 2-3 wt. % of a binder relative to a weight of the mixed raw material, respectively, into the mixed raw material to prepare a mixture in a suspension state;
a granulation process of a ferrite raw material, which comprises dry-spraying the mixture in a suspension state to form a granular shape;
a heat treatment and sintering process of heating the granular ferrite raw material at a temperature of 1,000-1,400° C. for 3-8 hours to calcine a ferrite sintered body;
a powdering process of the heat-treated ferrite sintered body to pulverize the same into powders;
a liquefaction process of the ferrite sintered body, which comprises mixing 40-60 wt. % of the powdered ferrite sintered body with 40-60 wt. % of water, adding 2-6 wt. % of a natural mordant to a weight of the mixture of ferrite powders and water, and ageing the same for 12-24 hours; and
a printing process of printing the liquefied ferrite sintered body onto a fabric in predetermined design and pattern.

2. The functional patch according to claim 1, wherein the series of processes comprise:
a ceramic mixing process of mixing ceramic powders to the powdered ferrite composition, wherein 20-30 wt. % of elvan, 20-30 wt. % of tourmaline, 10-18 wt. % of feldstar, 10-18 wt. % of kaolin, 6-10 wt. % of rock material, 6-10 wt. % of white clay and 4-8 wt. % of sericite are mixed together to prepare a mixed ceramic raw material;
a viscosifying process of the ceramic raw material, which comprises introducing 100-140 wt. %) of water to a weight of the mixed raw material, introducing 5-7 wt. % of limestone, 4-6 wt. % of titanium oxide, 6-8 wt. % of aluminum oxide and 1-3 wt. % of zirconium to a weight of the mixed raw material, and then, heating the mixture at a temperature of 200-300° C. for 3-8 hours to prepare a mixture in a slurry state;
a heat treatment and sintering process of heating the slurry type stirred product at a temperature of 1,000-1,400° C. for 3-8 hours to calcine a ceramic sintered body;
a powdering process of the heat-treated ceramic sintered body to pulverize the same into powders;
a liquefaction process of the ceramic sintered body, which comprises mixing 40-60 wt. % of the powdered ceramic sintered body with 40-60 wt. % of water, adding 2-6 wt. % of a natural mordant to a weight of the mixture of ceramic powders and water, and ageing the same for 12-24 hours; and
a printing process of printing the liquefied ceramic sintered body onto a fabric in predetermined design and pattern, thereby forming the functional patch.

3. The functional patch according to claim 2, wherein the series of processes comprise:
a mixing process of ferrite and ceramic which comprises mixing 40-60 wt. % of ceramic powders with 40-60 wt. % of the powdered ferrite composition;
a stirring process of ferrite and ceramic which comprises introducing 35-55 wt. % of water to a weight of the mixed ferrite and ceramic raw material to prepare a mixture in a paste state;
a heat treatment and sintering process of heating the stirred product of the paste type ferrite and ceramic at a temperature of 1,000-1,500° C. for 10-14 hours to calcine a sintered body of ferrite and ceramic;
a powering process of the ferrite and ceramic sintered bodies, which comprises pulverizing the heat-treated ferrite and ceramic into powders;
a liquefaction process of the ferrite and ceramic sintered bodies, which comprises mixing 40-60 wt. %) of the powdered ferrite and ceramic sintered bodies with 40-60 wt. % of water, adding 2-6 wt. % of a natural mordant to a weight of a mixture of the ceramic powders and water, and ageing the same for 12-24 hours; and
a printing process of printing the liquefied ferrite and ceramic sintered bodies onto a fabric in predetermined design and pattern, thereby forming the functional patch.
